(19) United States
(12) Patent Application Publication
(10) Pub. No.: US 2016/0280743 A1
(43) Pub. Date: Sep. 29, 2016

(54) BACTERIAL MUTANTS AND METHODS OF USE

(71) Applicant: The Board of Regents of the University of Oklahoma, Norman, OK (US)

(72) Inventors: Helen Zgurskaya, Norman, OK (US); Valentin Rybenkov, Norman, OK (US); Ganesh Krishnamoorthy, Norman, OK (US); David Wolloscheck, Norman, OK (US)

(21) Appl. No.: 15/081,021

(22) Filed: Mar. 25, 2016

Related U.S. Application Data
(60) Provisional application No. 62/138,781, filed on Mar. 26, 2015.

(51) Int. Cl. C07K 14/21; C07K 14/245; C12Q 1/18

(52) U.S. Cl. CPC C07K 14/21; C12Q 1/18; C07K 14/245; C07K 14/212

(57) ABSTRACT

Mutants of Gram-negative bacteria having outer membranes comprising modified FhuA nanopores absent an N-terminal plug domain are disclosed. The modified FhuA nanopores confer the outer membrane with enhanced permeability.

### CROSS REFERENCE TO RELATED APPLICATIONS/INCORPORATION BY REFERENCE STATEMENT

The present patent application claims priority to U.S. Provisional Patent Application Ser. No. 62/138,781, filed on Mar. 26, 2015, the entire contents of which is hereby expressly incorporated herein by reference.

### STATEMENT REGARDING FEDERALLY SPONSORED RESEARCH OR DEVELOPMENT

This invention was made with government support under Grant Number HDTRA1-14-1-0019-P00002, awarded by the Defense Threat Reduction Agency of the Department of Defense. The government has certain rights in the invention.

### BACKGROUND

Conventional antibiotic discovery efforts are focused on screening large libraries of chemical compounds and natural products to identify compounds that inhibit the bacterial growth or kill the bacteria. Gram-negative bacteria are notoriously difficult to target for drug screening and development because of the low permeability of their two-membrane cell envelopes. This permeability barrier is a result of synergistic actions of drug efflux transporters of various biochemical properties and the outer membrane barrier significantly restricting the uptake of various compounds based on their size and physico-chemical properties. The current approach to sensitize Gram-negative bacteria for drug discovery and development purposes is to mutationally inactivate drug efflux transporters. Such inactivation leads to significant sensitization of cells to biologically active compounds but markedly complicates the characterization of their mechanisms of action, due to the non-specific physiological effects of efflux inactivation, and further development of hits, due to the unknown mechanism of their penetration. We and others demonstrated that the major mechanism of antibiotic resistance in Gram-negative bacteria is the low permeability barrier, which is created by synergistic action of the active efflux transporters and the slow passive uptake across the outer membrane.

E. coli and P. aeruginosa are well-characterized model human pathogens studied extensively for their roles in clinical settings and antibiotic resistance mechanisms. The two species differ significantly in their susceptibilities to a broad range of antibiotics, with P. aeruginosa on average at least 10 fold more resistant to various antibiotics than E. coli. The major reason for such differences are believed to involve the outer membrane structure and composition, as well as the arsenal of efflux pump.

The E. coli outer membrane contains on average about 200,000 copies of general porins OmpF and OmpC, which provide the major path for hydrophilic and amphiphilic molecules with masses of up to ˜650 Da to cross the outer membrane. The hydrophobic molecules are thought to diffuse across the LPS-phospholipid bilayer. This diffusion is very slow because of the rigidity of the LPS-containing bilayer, which is electrostatically stabilized by divalent cations. The inner membrane of E. coli contains several multidrug efflux pumps, which differ in structure and mechanisms. The major efflux pump responsible for the intrinsic resistance of E. coli to antibiotics is AcrAB-TolC. In this pump, AcrB is the Resistance-Nodulation-Division (RND) transporter, which binds its substrates in the periplasm and expels them across the outer membrane with the help of AcrA, a periplasmic Membrane Fusion Protein (MFP), and TolC, an outer membrane channel. To enable such transport, these three components assemble a trans-envelope complex spanning both membranes of E. coli. Mutational inactivation of any of the three AcrAB-TolC components sensitizes E. coli to a variety of antibiotics. However, the E. coli genome encodes other close homologs of AcrB, which could be overproduced in cells lacking acrB. In contrast, TolC is a universal outer membrane channel, which is required for functions of at least nine different E. coli transporters involved in efflux of antibiotics and specific metabolites. Deletion of to/C inactivates all these transporters without a possibility of selection for suppressors.

The P. aeruginosa outer membrane does not contain general porins homologous to E. coli OmpF/C. The most abundant (about 200,000 copies per cell) outer membrane protein of this species is OprF, an outer membrane porin playing a structural role and providing a diffusion path for molecules less than ˜200 Da. The lack of large porins significantly diminishes the permeability of P. aeruginosa outer membrane. In contrast, P. aeruginosa cells carrying the plasmid-encoded E. coli OmpF are hypersusceptible to hydrophilic penems but not to other tested antibiotics. In addition, there are structural differences between E. coli and P. aeruginosa LPS components, which include the number and length of acyl chains of lipid A moieties and modifications in the LPS core and O-chains. As E. coli, P. aeruginosa cells produce one major efflux pump MexAB-OprM, which is highly homologous to AcrAB-TolC, has a similarly broad substrate specificity and shares the molecular mechanism. Unlike E. coli, there are twelve MexAB-OprM homologs encoded in the P. aeruginosa genome and most of them are co-expressed with their specific outer membrane channels homologous to OprM. Some of the outer membrane channels could be interchanged between the pumps, further complicating mutational inactivation of the efflux capacity of this bacterium.

It is well-recognized that the synergistic action of the low permeability barrier of the outer membranes and active drug efflux define the differences in susceptibilities of E. coli and P. aeruginosa to antibiotics. However, the permeation of different classes of antibiotics is affected by slow uptake and active efflux to different degrees. Presently, no rules exist to predict whether increasing uptake or reducing efflux would be the most efficient way to increase the potency of a specific class of compounds. Furthermore, there is a critical gap in knowledge about physicochemical properties and specific functional groups of compounds that define their permeation across cell walls of Gram-negative pathogens. Bacterial mutants useful in such investigational efforts and in drug screening would be desirable.

### BRIEF DESCRIPTION OF THE DRAWINGS

FIG. 1 shows the expression and localization of an open variant of FhuA membrane pores in E. coli WT-Pore and P. aeruginosa PAO1-Pore mutant strains. A. WT-Pore cells were grown to exponential phase in the presence and absence of 0.1% arabinose. The outer membrane fractions were isolated by differential detergent extraction and proteins analyzed by SDS-PAGE followed by immunoblotting with anti-His antibody. WM—whole membranes, IM—inner membrane fraction, OM—outer membrane fraction. B. PAO1 and PAO1-Pore cells were grown to exponential phase in the presence of 0.1 mM IPTG. The outer membrane fractions were isolated by differential detergent extraction and loaded onto a Ni²⁺ affinity column to purify FhuA ΔC/Δ4L protein. The elution fractions were analyzed by SDS-PAGE followed by immunoblotting with anti-His antibody.

FIG. 2 shows the effect of the pore expression on growth of E. coli and P. aeruginosa cells with different efflux capacities and permeability barriers. Growth of E. coli (A) and P. aeruginosa (B) cells in the presence of increasing concentrations of L-arabinose and IPTG, respectively. (Left panels) Overnight cultures were diluted 1:100 into a fresh LB medium supplemented with indicated concentrations of inducers. The cells were grown for 18 hours and OD₆₀₀ measured every 30 min. The OD₆₀₀ values collected at 18 hrs of incubation are plotted as a function of the inducer concentration in the medium. The middle and right panels show growth curves of E. coli (A) and P. aeruginosa (B) cells grown in the absence and presence of 0.1% arabinose and 0.1 mM IPTG, respectively.

FIG. 3 shows the effect of the pore expression on permeability of the outer membrane. Vancomycin susceptibility spot assay. Indicated E. coli (A) and P. aeruginosa (B) strains were seeded onto LB agar plates, ˜10⁸ cells per plate, with and without inducers 0.1% arabinose and 0.1 mM IPTG, respectively. Paper discs contained 100 μg or 200 μg of vancomycin for E. coli and P. aeruginosa, respectively. Seeded plates were incubated overnight at 37° C. Large clearance zones can be clearly seen in the presence of inducers.

FIG. 4 shows inducer-dependent changes of the erythromycin activity and uptake in various E. coli mutant cells with different efflux capacities and permeability barriers. A. Minimum inhibitory concentrations (MICs) of erythromycin in the E. coli cells grown in the presence of increasing concentrations of L-arabinose. Overnight cultures were diluted 1:100 into a fresh LB medium supplemented with indicated concentrations of the inducer and two-fold dilutions of erythromycin. The cells were incubated for 18 hours and OD₆₀₀ measured. The MIC values are plotted as a function of the inducer concentration in the medium. B. The time course of uptake of the radioactively-labeled [¹⁴C]-erythromycin into indicated E. coli cells grown in the presence of 0.1% arabinose.

FIG. 5 shows pore-dependent changes of macrolides activities and azithromycin uptake in mutant P. aeruginosa cells having different efflux capacities and permeability barriers. A. MICs of erythromycin in P. aeruginosa cells grown in the presence of increasing concentrations of IPTG plotted as a function of the inducer concentration in the medium. B. MICs of azithromycin in P. aeruginosa cells grown in the presence of increasing concentrations of IPTG plotted as a function of the inducer concentration in the medium. C. The time course of uptake of azithromycin into indicated P. aeruginosa cells grown in the presence of 0.1 mM IPTG as determined by LC-MS analyses.

FIG. 6 shows drug screening results for the potentiation of novobiocin and levofloxacin activity in strains of E. coli and P. aeruginosa, respectively, using several product libraries.

### DETAILED DESCRIPTION

Disclosed herein are novel bacterial mutants which have been sensitized, for example for drug discovery, drug screening, structure-activity relationships and development purposes. More particularly, the present disclosure includes embodiments of Gram-negative bacteria which have been sensitized to biologically active compounds by introducing modified nanopores in the outer membranes thereby modulating permeability properties of the outer membranes without compromising active efflux. For example, certain embodiments of the present disclosure are directed to Escherichia coli and Pseudomonas aeruginosa strains, as well as other strains, that are sensitized to antibiotics in a controlled manner due to increased rates of antibiotic uptake without compromising the active efflux and cell viability. This was carried out by inserting into bacterial chromosomes genes encoding modified protein nanopores. These nanopores can be produced in a tightly controlled manner so that different numbers of nanopores are inserted into outer membranes depending on the concentration of an inducer present in the external medium. In at least one non-limiting embodiment, the nanopore is FhuA ΔC/Δ4L, a genetically modified FhuA variant without its N-terminal plug (cork) domain and without four of its large external loops. Once in the outer membrane, these nanopores remove restrictions on the size and physico-chemical properties of compounds that can penetrate the outer membrane without compromising drug efflux activities or physiological states of cells. We demonstrated the modulation of the outer membrane permeability by: (i) measuring changes in susceptibilities of bacterial cells with nanopores to antibiotics as a dependence on the concentration of an inducer; (ii) measuring accumulation of radioactive chemicals of different sizes and fluorescent probes in cells containing nanopores. Described herein is the development of bacterial strains, in which permeation properties of the outer membrane and hence uptake of antibiotics and other molecules can be controlled.

Before describing various embodiments of the present disclosure in more detail by way of exemplary description, examples, and results, it is to be understood that the present disclosure is not limited in application to the details of methods and compositions as set forth in the following description. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that other embodiments of the inventive concepts may be practiced without these specific details. In other instances, features which are well known to persons of ordinary skill in the art have not been described in detail to avoid unnecessary complication of the description.

All of the compositions and methods of production and application thereof disclosed herein can be made and executed without undue experimentation in light of the present disclosure. While the compositions and methods of the present disclosure have been described in terms of particular embodiments, it will be apparent to those of skill in the art that variations may be applied to the compositions and/or methods and in the steps or in the sequence of steps of the method described herein without departing from the concept, spirit and scope of the inventive concepts. All such similar substitutes and modifications apparent to those of skilled in the art are deemed to be within the spirit, scope and concept of the present disclosure as described herein.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular. As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof′ is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the term “about” is used to indicate that a value includes the inherent variation of error for the composition, the method used to administer the composition, or the variation that exists among the study objects. Further, in this detailed description and the appended claims, each numerical value (e.g., temperature or time) should be read once as modified by the term “about” (unless already expressly so modified), and then read again as not so modified unless otherwise indicated in context. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

Also, any range listed or described herein is intended to include, implicitly or explicitly, any number within the range, particularly all integers, including the end points, and is to be considered as having been so stated. For example, “a range from 1 to 10” is to be read as indicating each possible number, particularly integers, along the continuum between about 1 and about 10. Thus, even if specific data points within the range, or even no data points within the range, are explicitly identified or specifically referred to, it is to be understood that any data points within the range are to be considered to have been specified, and that the inventors possessed knowledge of the entire range and the points within the range.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1). Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 1-20, 10-50, 50-100, 100-1,000, 1,000-3,000, 2,000-4,000, etc.

Where used herein in reference to a bacterium, the term “mutant” is intended to refer to a bacterium comprising a mutation in a “wild-type” or parental bacterium. “Wild-type” refers to the typical form (genotype and/or phenotype) of a bacterium, gene, nucleic acid, or protein as it occurs in nature and/or is the most common form in a natural population. In reference to a gene or nucleic acid, the term “mutation” refers to a gene or nucleic acid comprising an alteration in the wild type, such as but not limited to, a nucleotide deletion, insertion, and/or substitution. A mutation in a gene or nucleic acid generally results in either inactivation, decrease in expression or activity, increase in expression or activity, or another altered property of the gene or nucleic acid in the mutant bacterium comprising the mutation. In reference to a protein, the term “mutation” refers to protein comprising an alteration in the wild type, such as but not limited to, an amino acid deletion, insertion, and/or substitution. A mutation in a protein generally results in either inactivation, decrease in activity or effect, increase in activity or effect, or another altered property or effect of the protein in the mutant bacterium comprising the mutation. A mutant bacterium may comprise a gene or nucleic acid comprising a mutation. A mutant bacterium may also comprise a deletion of one or more entire genes, the deletion of the one or more genes comprising the mutation in the mutant bacterium. A mutant bacterium may also comprise an insertion of one or more additional genes, the insertion of the one or more additional genes comprising the mutation in the mutant bacterium. The additional one or more genes may be duplicates of a native gene already present in the wild-type bacterium, or may be non-native genes. A mutant bacterium may also comprise a substitution of one or more native genes by one or more non-native genes or mutated genes, the substitution comprising the mutation in the mutant bacterium

The novel constructed bacterial mutants and strains of the present disclosure can be used for example in methods comprising, but not limited to: (i) high-throughput screening programs to identify compounds with anti-bacterial activities; (ii) counter-screens to separate contributions of active efflux and uptake to a given compound cell permeation and accumulation; (iii) lead development efforts to build structure-activity relationships separately for active efflux and uptake for a given compound; (iv) improvement of the intracellular compound accumulation without inhibiting efflux; and (v) improvement of the intracellular compound accumulation by bypassing efflux pumps.

The sensitization/permeabilization methods of the present disclosure can be applied to all Gram-negative bacteria containing a typical outer membrane composed of lipids, lipopolysaccharides and porins, including but not limited to those shown below in Table 1, which is a list of clinically important human pathogens that are most commonly targeted in current drug discovery programs.

TABLE 1
Examples of bacteria which can be modified as described herein.

```
  1. Escherichia coli and other Escherichia species (spp)
  2. Klebsiella pneumoniae and Klebsiella spp.
  3. Salmonella enterica and Salmonella spp.
  4. Enterobacter cloacea and Enterobacter spp.
  5. Burkholderia cenocepacia complex
  6. Burkholderia thailandensis and other Burkholderia spp.
  7. Acinetobacter baumannii
  8. Pseudomonas aeruginosa and Pseudomonas spp.
  9. Yersinia pestis and Y. pneumoniae
10. Shigella spp.
11. Francisella tularensis and Francisella spp.
12. Borrelia spp.
13. Niesseria meningitidis and N. gonorrhoeae
14. Serratia spp.
15. Proteus mirabilis and Proteus spp.
16. Haemophilus influenza and Haemophilus spp.
17. Vibrio cholera and Vibrio spp.
18. Citrobacter spp.
19. Bacteroides fragilis and Bacteroides spp.
```

Several embodiments of the present disclosure, having now been generally described, will be more readily understood by reference to the following examples and embodiments, which are included merely for purposes of illustration, and are not intended to be limiting. The following detailed examples of the present disclosure are to be construed, as noted above, only as illustrative, and not as limitations of the embodiments described herein in any way whatsoever. Those skilled in the art will promptly recognize appropriate variations from the various compositions, structures, components, procedures and methods.

To control the permeability of the outer membrane of the bacterial mutants of certain embodiments of the present disclosure, we incorporated into bacterial chromosomes a gene encoding an open variant of FhuA. Wild type FhuA forms a 22-stranded beta-barrel in the outer membrane of E. coli. The non-specific transport through such a structure is prevented by a plug (“cork”) domain. Removal of this plug creates a large hole in the outer membrane. U.S. Pat. No. 8,916,684 describes a genetically modified FhuA variant without its N-terminal plug (cork) domain and without four of its external loops. The variant is referred to as FhuAΔC/Δ4L, and is encoded in a plasmid pPR-IBA1. When inserted into the membrane of E. coli, FhuAΔC/Δ4L form large nanopores. Any open FhuA variant or FhuA homolog which functions in accordance with the requirements of the present disclosure may be used. Other mutations (e.g., deletions, substitutions, or insertions) may be made in addition to or instead of the FhuA mutation to form novel bacterial strains of the bacterial species described herein, including but not limited to mutations in the genes shown in Table 2.

TABLE 2
Examples of genes that can be mutated in nanopore-variant bacterial mutants.

| | | | General and specific porins |
|---|---|---|---|
| | Outer membrane | Efflux pump for gene | for inactivation in |
| Bacterial | pores for insertion | deletions in combination | combination with pores and |
| species | onto chromosome | with pores | pumps |
| Burkholderia | OrbA ΔC/Δ4L | amrRAB-oprA, bpeAB- | ompA, opcP1, opcP2 |
| cepacia | | oprB, bpeEF-oprC | |
| Burkholderia | | | |
| thailandensis | | | |
| Acinetobacter | FhuA ΔC/Δ4L | adeAB, adeFGH, adeIJK | ompA, carO, oprD |
| baumannii | | | |
| Pseudomonas | FhuA ΔC/Δ4L | mexAB-oprM, mexCD- | oprD |
| aeruginosa | | oprJ, mexEF-oprN, | |
| | | mexXY, mexGHI, triABC, | |
| | | opmH, mexJKL | |
| Escherichia coli | FhuA ΔC/Δ4L | acrB, acrD, acrEF, emrB, | ompF, ompC, |
| and other | | emrY, entS, macB, mdtC, | |
| enterobacteria | | mdtF, tolC, mdfA, emrE, | |
| | | norM | |

### EXPERIMENTAL

### Microbiological Assays

Susceptibilities of E. coli and P. aeruginosa cells to different classes of antibiotics were determined by two-fold broth dilution method [1] with following modifications. Cells were grown in Luria-Bertani (LB) broth (tryptone 10 g/L, yeast extract 5 g/L and NaCl 5 g/L) with appropriate selection markers as needed, at 37° C. with shaking at 200 rpm. When needed, at OD₆₀₀˜0.3, either L-arabinose (final concentration 0.1%) or IPTG (final concentration 0.1 mM) were added to induce the expression of the pore and cells were further incubated until OD₆₀₀ reached 1.0. MICs of various antimicrobial agents were measured in 96-well micro-titer plates. For this purpose, exponentially growing cells were inoculated at a density of 10⁵ cells/ml into wells containing LB medium in the presence of two-fold increasing concentrations of drugs under investigation at constant inducer concentration of 0.1% arabinose for E. coli or 0.1 mM IPTG for P. aeruginosa strains. Cell growth was determined visually or using Spark 10M microplate reader (Tecan) after incubation of the micro-titer plates at 37° C. for 16 h.

Strains and Plasmids

Non-limiting examples of P. aeruginosa, E. coli, and Acinetobacter baumannii strains and plasmids are listed in Table 3. To construct pGK-LAC-fhuAΔC/Δ4L (Gm^{r}), the fhuAΔC/Δ4L gene was amplified from the pPR-IBA1-FhuA ΔC/Δ4L plasmid and ligated into the pUC18-mini-Tn7T-LAC suicide delivery vector restricted with SacI and KpnI enzymes. Gentamicin (15 μg/ml) was used for selection. The pGK-araC-P_{BAD}-fhuAΔC/Δ4L (Tp^{r}) plasmid was constructed by amplifying and cloning the fhuAΔC/Δ4L gene into NcoI and EcoRI restriction sites of the pUC18-miniTn7T-araC-P_{BAD} (pTJ1) suicide delivery vector. Trimethoprim (500 μg/ml) was used for selection.

The insertion of fhuAΔC/Δ4L onto the E. coli chromosome requires a mini-Tn7T suicide delivery vector carrying R6K origin of replication that will not self-replicate in E. coli cells. To construct pR6KT-mini-Tn7T-araC-pBAD-(Km^{r}) and pR6KT-mini-Tn7T-LAC-(Km^{r}), we amplified lacI^{q}-the LAC-FhuAΔC/Δ4L fragment (3023 bp) using the pGK-LAC-fhuAΔC/Δ4L plasmid as a template and the lacI^{q}-P_{TAC}-MCS fragment (1497 bp) without an insert from the pUC18-mini-Tn7T-LAC plasmid. Subsequently, the pUC18TR6K-miniTn7T vector was treated either with NsiI/KpnI or NsiI/SacI enzymes. The resulting NsiI/KpnI or the NsiI/SacI fragments of the vector were ligated with the lacI^{q}-P_{TAC}-FhuAΔC/Δ4L PCR product treated with NsiI and KpnI enzymes or with the NsiI/SacI treated lacI^{q}-P_{TAC}-MCS fragment.

For the construction of pR6KT-mini-Tn7T-araC-pBAD plasmid, two different PCRs were done. PCR #1: araC-P_{BAD}-MCS fragment and araC-pBAD-fhuAΔC/Δ4L was amplified using pUC18-mini-Tn7T-araC-P_{BAD} or pUC18-mini-Tn7T-araC-P_{BAD}-FhuAΔC/Δ4L as templates. The pR6KT-mini-Tn7T backbone was amplified using the R6K mini

Tn7T vector as a template. The PCR products were cut with AscI and NotI enzymes and ligated with each other.

Insertion of fhuAΔC/Δ4L (Km^{r}) onto E. coli and P. aeruginosa chromosomes was achieved as described in [2]. Briefly, the respective suicide delivery vectors described above carrying the FhuAΔC/Δ4L gene was electroporated along with pTNS2 helper plasmid and grown for 1 h in LB medium containing 20 mM glucose. The cells were then plated onto LB agar containing respective antibiotics for the selection: kanamycin (25 μg/ml) for E. coli and gentamicin (30 μg/ml) or trimethoprim (500 μg/ml for PAO1 WT and 30 μg/ml for Δ3 strain) for P. aeruginosa and incubated for 16 h at 37° C.

Macrolides Uptake

E. coli cells were subcultured from stationary phase 1:100 into a fresh LB medium. Expression of FhuAΔC/Δ4L was induced at OD₆₀₀ of 0.3 with 0.1% arabinose and, subsequently, cells were grown to OD₆₀₀ of 1. Cells were collected via centrifugation and pellets were resuspended in PMG buffer (50 mM potassium phosphate, 1 mM magnesium sulfate and 0.4% glucose at pH 7.0) at one-tenth the original culture volume.

Uptake assay were performed in a 96-well MultiScreenHTS FB Filter Plates (1.0/0.65 μm; EDM Millipore). For uptake of C-14 labeled erythromycin (PerkinElmer), antibiotic was added to 1 ml of concentrated cells to a final concentration of 10 μM and a specific activity of 0.025 Ci/mmol. 100 μl aliquots were taken at the indicated time points and applied to 96 well filter plates connected to a HTS Vacuum Manifold (EMD Millipore). The filters were allowed to dry and radioactivity was detected using a Tri-Carb 2810TR scintillation counter (PerkinElmer). Intracellular concentrations were corrected to the amounts of erythromycin accumulated on filters at 0.5 min time point. Concentrations were calculated assuming that an OD₆₀₀ of 1 contains 1×10⁹ cells of E. coli per milliliter with an average cell volume of 1 μm³.

For LC-MS analyses, P. aeruginosa cells were grown to stationary phase in 50 mM MOPS (pH 7.2) M9 minimal medium supplemented with 1% glycerol and trace ions at 37° C. Stationary phase cells were subcultured 1:100 for 16 hours in the same fresh medium at 37° C. After 16 hours, cultures were induced with 0.1 mM IPTG for 4 hours at 37° C. Cells were collected by centrifugation at room temperature and resuspended in PMG buffer at one-tenth the original culture volume. Azithromycin (final concentration of 5 μM) was added to 500 μl of cells (OD˜12-15). At appropriate time points, 100 μl of cells were removed and collected onto filter plates using a HTS Vacuum Manifold (EMD Millipore). Filters were dried overnight and extracted in a two-step process using 1) 800 μl of 100% HPLC grade methanol and 2) 200 μl of 80% HPLC methanol: 20% ultrapure water. Extracts from the two steps were combined and analyzed by HPLC (ACQUITY UPLC BEH C18 pre-column (1.7 uM, 2.1×5 mm), ACQUITY UPLC BEH C18 column (1.7 uM, 2.1×100 mm); Waters) followed by MS (QTOF Agilent Accurate Mass High Resolution; Agilent Technologies). For HPLC, two mobile phases (A—100% water, B—100% acetonitrile; both with 0.1% formic acid) were used with a step gradient of 20, 50 and 100% of B for 1, 4, and 11 min, respectively. We determined that for all four PAO1, PAO1-Up, Δ3, Δ3-Up P. aeruginosa strains the number of cells in 1 OD to be 1.5×10⁹. The intracellular volume was assumed to be 1 μm³ [3]. Intracellular concentrations were corrected to the amounts of azithromycin accumulated in non-permeable PAO1 cells.

Results

Genes Encoding Recombinant Protein Pores can be Introduced into Various Gram-Negative Bacteria.

As noted above, to control the permeability of outer membranes, we incorporated onto bacterial chromosomes a gene encoding an open variant of FhuA, FhuAΔC/Δ4L, which when purified and reconstituted into liposomes forms a pore with an internal diameter of ˜2.4 nm. We constructed E. coli strains carrying the plasmid-borne and chromosomally encoded FhuAΔC/Δ4L pore under the control of IPTG and arabinose-inducible promoters, and in genetic backgrounds with the wild-type repertoire of efflux pumps (WT-Pore), lacking the universal TolC channel required for activities of various transporters (TolC-Pore) or lacking genes encoding all nine TolC-dependent efflux pumps (Table 3). The constructed P. aeruginosa strains produced a chromosomally encoded FhuAΔC/Δ4L under control of IPTG- and arabinose-inducible promoters and contained either the full array of efflux pumps (PAO1-Pore), lacked the three major RND-type transporters MexAB, MexCD and MexXY (Δ3-Pore), lacked four RND efflux pumps (ΔmexAB-oprM ΔmexCD-oprJ ΔmexJK ΔmexXY), or lacked six RND efflux pumps (ΔmexAB-oprM ΔmexCD-oprJ ΔmexEF-oprN ΔmexJK ΔmexXY ΔtriABC) (Table 3). The constructed Acinetobacter baumannii strains produced a chromosomally encoded FhuAΔC/Δ4L under the control of IPTG- and arabinose-inducible promoters. The results below are shown only for E. coli WT-Pore and TolC-Pore and their parental strains and for P. aeruginosa PAO1-Pore and Δ3-Pore and their parental strains but the same is also true for other constructed strains.

TABLE 3
Plasmids and strains

| Strains | Description | Source |
|---|---|---|
| pUC18-mini-Tn7T- | A suicide delivery vector | Damron and |
| LAC-(Gm^{r}) | | Shweizer |
| | | et al. 2013 |
| pUC18-mini-Tn7T- | A suicide delivery vector | Choi and |
| araC-P_{BAD}-(Tp^{r}) | | Schweizer |
| | | 2006 |
| pUC18-mini-Tn7T- | mini-Tn7T-Gm^{r}-lacI^{q}-pLAC vector containing fhuA | This study |
| LAC-FhuA ΔC/Δ4L | ΔC/Δ4L gene | |
| (Gm^{r}) | | |
| pUC18-mini-Tn7T- | mini-Tn7T-Tp^{r}-araC-P_{BAD} vector containing fhuA | This Study |
| araC-P_{BAD}-FhuA | ΔC/Δ4L gene | |
| ΔC/Δ4L (Tp^{r}) | | |
| pCF430-araC-P_{BAD} | A low-copy E. coli expression vector, Tc^{r} | Newman and |
| | | Fuqua 1999 |
| pPR-IBA1-FhuA | pET-based plasmid containing fhuA ΔC/Δ4L gene | Mohammad |
| ΔC/Δ4L | | and Movileanu |
| | | et al. 2011 |
| pCF-FhuA | pCF430 producing fhuA ΔC/Δ4L gene under an | This Study |
| | arabinose inducible promoter, Tc^{r} | |

Escherichia coli

| | | |
|---|---|---|
| BW 25113 (WT) | Wild-type strain Δ(araD-araB)567 Δ(rhaD-rhaB)568 | |
| | ΔlacZ4787 (::rrnB-3) hsdR514 rph-1 | |
| GD102 (ΔTolC) | BW25113 ΔtolC-ygiBC | |
| M6394 | Wild type GC4468 Δ(argF-lac)169 λ⁻ IN(rrnD- | Gift from |
| | rrnE)1 rpsL179(strR) | Judah Rosner |
| M6394Δ9 | M6394 ΔacrB ΔacrD ΔacrEF::spc ΔemrB ΔemrY | Gift from |
| | ΔentS::cam ΔmacB ΔmdtC ΔmdtF | Judah Rosner |
| M6394ΔtolC | M6394 ΔtolC | Gift from |
| | | Judah Rosner |
| GKCW101 (WT-Pore) | BW25113 attTn7::mini-Tn7T-Tp^{r}-araC-P_{araBAD}- | This study |
| | fhuAΔCΔ4L | |
| GKCW102 | BW25113 attTn7::mini-Tn7T-Tp^{r}-araC-P_{araBAD}- | This study |
| | MCS | |
| GKCW103 (ΔTolC- | GD102 attTn7::mini-Tn7T-Tp^{r}-araC-P_{araBAD}- | This study |
| Pore) | fhuAΔCΔ4L | |
| GKCW104 | GD102 attTn7::mini-Tn7T-Tp^{r}-araC-P_{araBAD}-MCS | This study |
| GKCW105 | M6394 attTn7::mini-Tn7T-Tp^{r}-araC-P_{araBAD}- | This study |
| | fhuAΔCΔ4L | |
| GKCW106 | M6394 attTn7::mini-Tn7T-Tp^{r}-araC-P_{araBAD}-MCS | This study |
| GKCW107 | M6394Δ9 attTn7::mini-Tn7T-Tp^{r}-araC-P_{araBAD}- | This study |
| | fhuAΔCΔ4L | |
| GKCW108 | M6394Δ9 attTn7::mini-Tn7T-Tp^{r}-araC-P_{araBAD}- | This study |
| | MCS | |
| GKCW109 | M6394ΔtolC attTn7::mini-Tn7T-Tp^{r}-araC-P_{araBAD}- | This study |
| | fhuAΔCΔ4L | |
| GKCW110 | M6394ΔtolC attTn7::mini-Tn7T-Tp^{r}-araC-P_{araBAD}- | This study |
| | MCS | |

Pseudomonas aeruginosa

| | | |
|---|---|---|
| PAO1 | Wild-type strain | Gift from O. Lomovskaya |
| PAO1Δ3 (Δ3) | PAO1 but ΔmexAB ΔmexCD ΔmexXY | This study |
| PAO1116 | PAO1 ΔmexAB-oprM ΔmexCD-oprJ ΔmexEF-oprN | Mima and |
| | ΔmexJK ΔmexXY ΔtriABC | Schweizer, |
| | | 2007 |
| PAO325 | PAO1 ΔmexAB-oprM ΔmexCD-oprJ ΔmexJK ΔmexXY | Chuanchue et |
| | | al, 2002 |
| GKCW111 | PAO1 attTn7::mini-Tn7T-Gm-lacI^{q}-pLAC-MCS | This study |
| GKCW112 | PAO1Δ3 attTn7::mini-Tn7T-Gm^{r}-lacI^{q}-pLAC-MCS | This study |
| GKCW113 | PAO1 attTn7::mini-Tn7T-Tp^{r}-araC-P_{araBAD}-MCS | This study |
| GKCW114 | PAO1Δ3 attTn7::mini-Tn7T-Tp^{r}-araC-P_{araBAD}- | This study |
| | MCS | |
| GKCW115 (PAO1- | PAO1 attTn7::mini-Tn7T-Gm^{r}-lacI^{q}-pLAC- | This study |
| Pore) | fhuAΔCΔ4L | |
| GKCW116 (Δ3-Pore) | PAO1Δ3 attTn7::mini-Tn7T-Gm^{r}-lacI^{q}-pLAC- | This study |
| | fhuAΔCΔ4L | |
| GKCW117 | PAO1 attTn7::mini-Tn7T-Tp^{r}-araC-P_{araBAD}- | This study |
| | fhuAΔCΔ4L | |
| GKCW118 | PAO1Δ3 attTn7::mini-Tn7T-Tp^{r}-araC-P_{araBAD}- | This study |
| | fhuAΔCΔ4L | |
| GKCW119 | PAO1116 attTn7::mini-Tn7T-Gm-lacI^{q}-pLAC-MCS | This study |
| GKCW120 | PAO1116 attTn7::mini-Tn7T-Gm^{r}-lacI^{q}-pLAC- | This study |
| | fhuAΔCΔ4L | |
| GKCW121 | PAO325 attTn7::mini-Tn7T-Gm-lacI^{q}-pLAC-MCS | This study |
| GKCW122 | PAO325 attTn7::mini-Tn7T-Gm^{r}-lacI^{q}-pLAC- | This study |
| | fhuAΔCΔ4L | |

Acinetobacter baumannii

| | | |
|---|---|---|
| ATCC 17976 | Wild-type strain | ATCC19606 |
| JWW101 | ATCC 17976 Str^{r} | This study |
| JWW102 | JWW1 attTn7::mini Tn7-Tp^{r}-lacI^{q}-P_{TAC}-MCS | This study |
| JWW103 | JWW1 attTn7::mini Tn7-Tp^{r}-lacI^{q}-P_{TAC}- | This study |
| | fhuAΔCΔ4L | |
| JWW104 | JWW1 attTn7::mini Tn7-Tp^{r}-araC-P_{BAD}-MCS | This study |
| JWW105 | JWW1 attTn7::mini Tn7-Tp^{r}-araC-P_{BAD}- | This study |
| | fhuAΔCΔ4L | |

Str^{r}—streptomycin resistance,
Gm^{r}—gentamycin resistance,
Tp^{r}—trimethoprim resistance,
Tc^{r}—tetracycline resistance

Recombinant Pores are Expressed in an Inducer-Dependent Manner and Localized to the Outer Membrane.

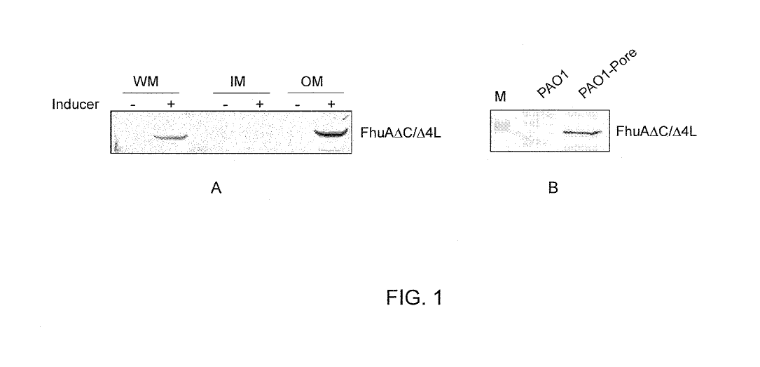

To confirm that the expressed FhuAΔC/Δ4L is properly localized, the total membrane fractions of E. coli WT-Pore and P. aeruginosa PAO1-Pore cells treated with inducers arabinose and IPTG, respectively, were isolated by differential ultracentrifugation and detergent extraction and analyzed by immunoblotting with monoclonal antibodies against the six-histidine affinity tag of FhuAΔC/Δ4L. A single 52 kDa band corresponding in size to FhuAΔC/Δ4L was detected in the outer membrane fractions of cells treated with inducers (FIG. 1). At 0.1% arabinose, E. coli cells carry approximately 30 pores per cell, whereas approximately 7 pores per cell are present in the outer membranes of P. aeruginosa cells induced with 0.1 mM IPTG.

TABLE 4
Vancomycin susceptibilities of E. coli and P. aeruginosa

| | Zone of clearance (mm) | | |
|---|---|---|---|
| Strain | No inducer | Arabinose (0.1%) | IPTG (0.1 mM) |
| WT | 5 | 6 | 6 |
| WT-Pore | 8 | 16 | 11 |
| ΔTolC | 5 | 6 | 5 |
| ΔTolC-Pore | 7 | 15 | 16 |
| PAO1 | 11 | 12 | 10 |
| PAO1-Pore | 12 | 21 | 19 |
| Δ3 | 10 | 12 | 9 |
| Δ3-Pore | 12 | 22 | 18 |

^{a}10 μl of 10 mg/ml solution of vancomycin (100 μg) for E. coli and 10 μl of 20 mg/ml of vancomycin (200 μg) for P. aeruginosa were spotted onto filter paper discs.

Gram-Negative Bacteria Carrying the Pores are Viable.

To investigate how permeabilization of the outer membrane affects susceptibility to antibiotics of efflux-proficient and efflux-deficient cells, the growth inhibition experiments and MICs measurements were carried out for P. aeruginosa and E. coli cells with and without the fhuAΔC/Δ4L gene on chromosomes and treated with increasing concentrations of the indicated inducer. For this purpose, induced cells were dispensed into 96-well microplates with LB medium containing two-fold dilutions of an antibiotic of interest and a respective concentration of an inducer.

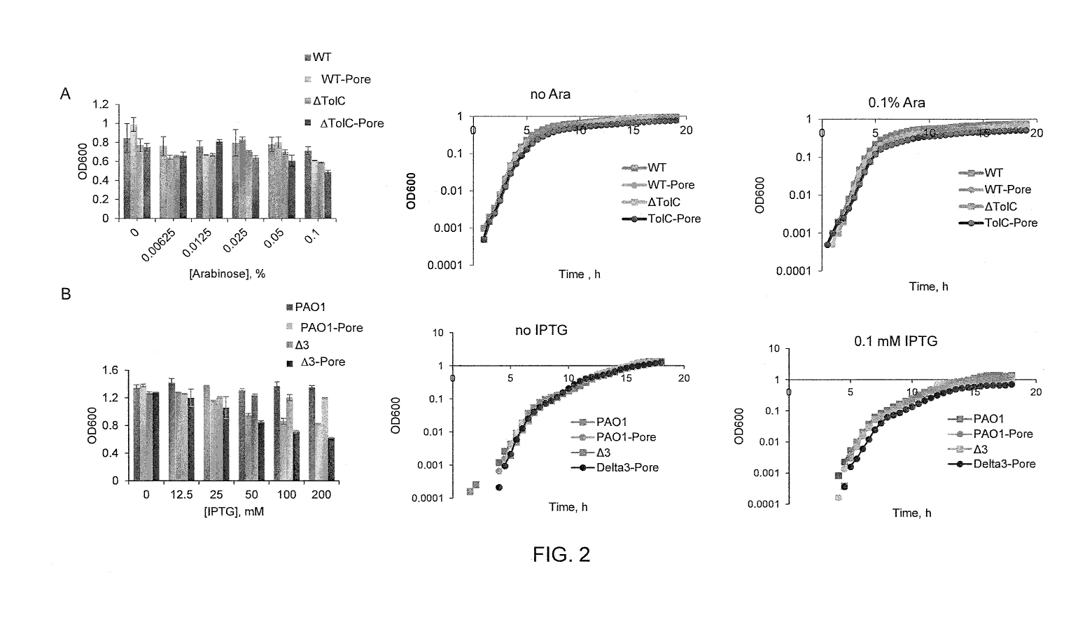

The increasing concentrations of inducers did not impede the growth rates of E. coli or P. aeruginosa in the absence of an antibiotic but triggered a somewhat early transition into a stationary phase, as seen from the reduction of OD_{600 by} ˜30-40% after 19 hours of incubation (FIG. 2). In P. aeruginosa cells, the effect of the inducer was visible only in cells carrying the fhuAΔC/Δ4L gene (FIG. 2B).

Pores Sensitize Gram-Negative Bacteria to Antibiotics that Cannot Permeate the Outer Membrane.

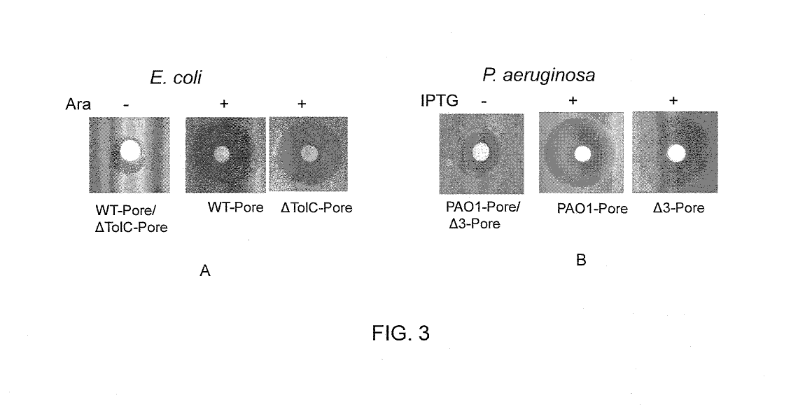

The inducer-controlled permeabilization of the outer membrane was first tested by a disc susceptibility assay with vancomycin, an antibiotic of ˜1450 Da in size that does not penetrate the outer membranes of Gram-negative bacteria. In the absence of an inducer, all strains remained resistant to vancomycin, but became hypersusceptible to vancomycin in the presence of the respective inducers (FIG. 3 and Table 4).

Permeabilization of the Outer Membrane Potentiates Activities of Antibiotics in Efflux-Proficient and Efflux-Deficient Cells.

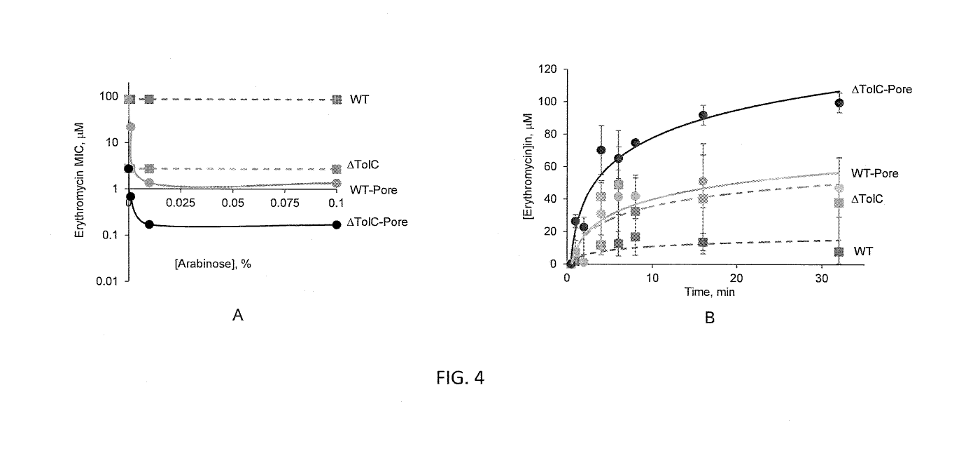

We next analyzed the growth inhibition in the presence of a macrolide antibiotic erythromycin with a mass of 734 Da, which exceeds the cut-offs of OmpF/C and OprF, the general porins of E. coli and P. aeruginosa, respectively. As expected the activity of erythromycin, a known substrate of efflux pumps, was significantly potentiated in efflux-deficient E. coli and P. aeruginosa cells (FIG. 3). We further found that in the strains carrying the pore gene, MICs of erythromycin depended on concentrations of inducers and hence on the expression levels of the pore. In agreement with previous studies, the MICs of erythromycin in E. coli WT and ΔTolC cells differed by 16 fold (87 μM and 5.5 μM for the WT and ΔTolC, respectively) and remained unchanged in the range of 0.001-0.5% arabinose concentrations (FIG. 4A). In contrast, the MICs of erythromycin in WT-Pore and ΔTolC-Pore cells decreased with increasing concentrations of the inducer and plateaued at 0.01% arabinose and at 1.4 μM and 0.17 μM of erythromycin, respectively (FIG. 4A). Thus, potentiation of the erythromycin activity increases with the increasing permeability of the outer membrane.

Permeabilization of the outer membrane in WT-Pore E. coli reduced the MIC of erythromycin by 64 fold, bringing it down to 1.4 μM, which is below the MIC in the ΔTolC cells. Unexpectedly, the MIC of erythromycin decreased by additional 16 fold in permeabilized ΔTolC-Pore cells, 0.17 μM, resulting in the total potentiation of erythromycin by 512 fold. This result suggested that permeabilization of the outer membrane of E. coli potentiates erythromycin activity independently whether the TolC-dependent efflux pumps are active or not. Furthermore, the growth inhibition of WT-Pore and ΔTolC-Pore was apparent even at sub-inhibitory concentrations of erythromycin used in experiments (FIG. 4A) suggesting that there is not a threshold barrier and erythromycin continuously accumulates in these cells.

To confirm that the changes in MICs reflect the changes in the intracellular accumulation of antibiotics, the uptake of the radioactively-labeled [¹⁴C]-erythromycin was analyzed in four E. coli strains. As shown on FIG. 4B, the levels of intracellular [¹⁴C]-erythromycin increase with time and are the highest in ΔTolC cells and the induced WT-Pore and ΔTolC-Pore cells.

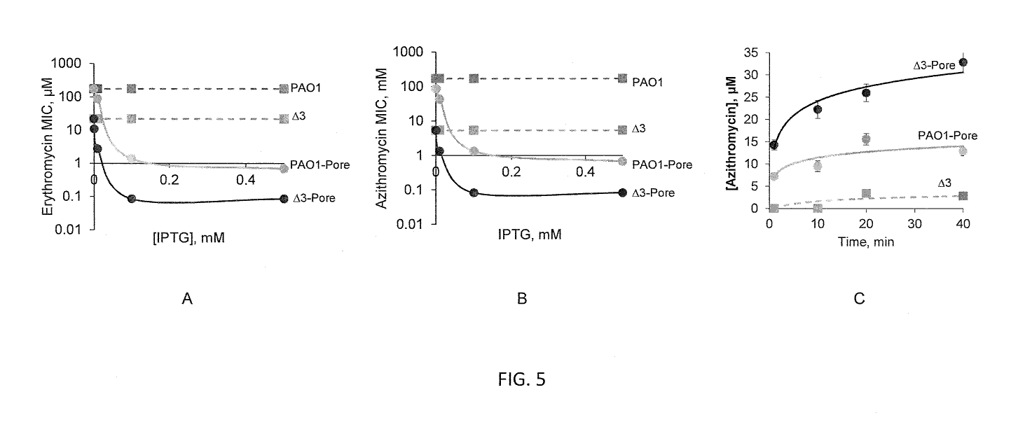

The erythromycin potentiation profile was similar in P. aeruginosa cells. The wild type PAO1 cells are more resistant to erythromycin than E. coli with MICs at 174 μM (Table 5 and FIG. 5). Inactivation of the three major efflux pumps leads to 8-fold decrease in susceptibility of Δ3 cells to erythromycin with MIC at 21.8 μM. As with E. coli cells, the increasing concentrations of the inducer IPTG did not affect the growth and MICs of erythromycin in PAO1 and Δ3 cells (FIG. 2). However, the presence of increasing concentrations of IPTG reduced the erythromycin MICs in PAO1-Pore cells and in Δ3-Pore cells (FIG. 5A). The MIC of erythromycin in PAO1-Pore and Δ3-Pore cells plateaued at the values of 1.4 μM and 0.085 μM, which corresponds to 128 and 256 fold drop in MICs, respectively.

TABLE 5
Minimal inhibitory concentrations of antibiotics and their selected physico-chemical properties, E. coli.

| | | | | | Fold MIC change | | | | | |
|---|---|---|---|---|---|---|---|---|---|---|
| | | | | | OM barrier | | Efflux | | | |
| | MIC (μg/ml) | | | | WT/ | ΔTolC/ | | WT-Pore/ | Properties | |
| | BW 25113 (WT) | | GD102 (ΔTolC) | | WT- | ΔTolC- | WT/ | ΔTolC- | | logD at |
| Drug | — | Pore | — | Pore | Pore | Pore | ΔTolC | Pore | Mass | pH = 7.4 |
| Amikacin | 2 | 2 | 2 | 2 | 1 | 1 | 1 | 1 | 585.6 | −15.1 |
| Gentamicin | 4 | 2 | 4 | 4 | 2 | 1 | 1 | 0.5 | 477.6 | −11.79 |
| Streptomycin | 2 | 2 | 4 | 2 | 1 | 2 | 0.5 | 1 | 581.57 | −12.16 |
| Levofloxacin | 0.031 | 0.016 | 0.004 | 0.004 | 2 | 1 | 8 | 4 | 361.37 | −0.28 |
| Nalidixic acid | 8 | 8 | 1 | 1 | 1 | 1 | 8 | 8 | 232.24 | −0.25 |
| Lincomycin | 512 | 512 | 64 | 64 | 1 | 1 | 8 | 8 | 406.54 | −0.99 |
| Chloramphenicol | 2 | 2 | 0.5 | 0.5 | 1 | 1 | 4 | 4 | 323.13 | 0.69 |
| Triclosan | 0.031 | 0.031 | 0.004 | 0.002 | 1 | 2 | 8 | 16 | 289.54 | 4.8 |
| Tetracycline | 0.5 | 0.25 | 0.125 | 0.125 | 2 | 1 | 4 | 2 | 467.52 | −13.63 |
| Ciprofloxacin | 0.016 | 0.004 | 0.002 | 0.002 | 4 | 1 | 8 | 2 | 331.34 | −0.81 |
| Proflavine | 32 | 32 | 8 | 8 | 1 | 1 | 4 | 4 | 209.25 | 0.94 |
| SDS | 10000 | 10000 | 5 | 5 | 1 | 1 | 2000 | 2000 | 288.38 | 2.04 |
| Cloxacillin | 512 | 128 | 1 | 1 | 4 | 1 | 512 | 128 | 435.88 | −0.98 |
| Carbenicillin | 4 | 0.5 | 1 | 0.5 | 8 | 2 | 4 | 1 | 378.4 | −5.91 |
| Ampicillin | 16 | 2 | 8 | 0.5 | 8 | 16 | 2 | 4 | 349.405 | −2.26 |
| Coumermycin | 8 | 2 | 8 | 0.5 | 4 | 16 | 1 | 4 | 1110.08 | 0.7 |
| Rifampicin | 4 | 0.25 | 4 | 0.25 | 16 | 16 | 1 | 1 | 822.94 | 2.76 |
| Vancomycin | 128 | 8 | 256 | 4 | 16 | 64 | 0.5 | 2 | 1449.25 | −4.86 |
| Erythromycin | 64 | 4 | 4 | 0.125 | 16 | 32 | 16 | 32 | 733.93 | 1.57 |
| Azithromycin | 2 | 0.5 | 0.5 | 0.031 | 4 | 16 | 4 | 16 | 748.98 | −1.23 |
| Virginiamycin | >256 | 256 | 8 | 0.5 | ≧2 | 16 | ≧64 | 512 | 525.59 | 2.38 |
| Novobiocin | 128 | 32 | 0.5 | 0.125 | 4 | 4 | 256 | 256 | 612.62 | 1.36 |

To determine whether the potentiation profile is representative for the class of macrolide antibiotics, we analyzed MICs and accumulation of azithromycin, a 15-membered macrolide with a mass of 749 Da. The effect of permeabilization of the outer membrane on the activity of azithromycin was even more dramatic. MICs of azithromycin decreased with increasing concentrations of the inducer in both PAO1-Pore and Δ3-Pore cells by 32 and 64 fold, respectively (FIG. 5B). As a result, the MIC of azithromycin decreased by more than 2000 fold from 171 μM in PAO1 cells to 0.083 μM in the induced Δ3-Pore cells (FIG. 5B). In agreement, LC-MS analyses of azithromycin uptake in P. aeruginosa cells showed that the permeabilized PAO1-Pore and Δ3-Pore cells accumulate azithromycin at levels significantly higher than PAO1 and Δ3 cells (FIG. 5C). Thus, the contributions of active efflux and slow uptake to antibiotic potency in cells vary significantly even with small differences in structure and properties of an antibiotic.

Permeabilization of the Outer Membrane Potentiates Activities of Various Groups of Antibiotics.

We next determined MICs for a broad range of anti-bacterial agents belonging to various classes of antibiotics and antimicrobial agents in E. coli and P. aeruginosa cells treated with fixed concentrations of inducers. Table 5 summarizes the MICs of antibiotics in E. coli and Table 6 in P. aeruginosa strains and their variants sensitized to antibiotics by the presence of FhuAΔC/Δ4L pores in their outer membranes. The tested antibacterials vary by their physico-chemical properties, target localizations and whether or not they are substrates of efflux pumps. The impact of the outer membrane permeabilization differs for E. coli and P. aeruginosa variants reflecting the differences in contributions of the outer membrane permeability barrier and drug efflux pumps to the intracellular accumulation of antibiotics between these species.

TABLE 6
Minimal inhibitory concentrations of antibiotics in P. aeruginosa.

| | | | | | Fold MIC change | | | | | |
|---|---|---|---|---|---|---|---|---|---|---|
| | | | | | OM barrier | | Efflux | | Physico-chemical | |
| | MIC (μg/ml) | | | | PAO1/ | Δ3/ | | PAO1- | properties | |
| | PAO1 | | Δ3 | | PAO1- | Δ3- | PAO1/ | Pore/Δ3- | | logD at |
| Drug | — | Pore | — | Pore | Pore | Pore | Δ3 | Pore | Mass | pH = 7.4 |
| Amikacin | 1 | 2 | 1 | 1 | 0.5 | 1 | 1 | 2 | 585.6 | −15.1 |
| Tobramycin | 2 | 1 | 0.5 | 0.5 | 2 | 1 | 4 | 2 | 444.44 | −3.7 |
| Coumermycin | 16 | 1 | 16 | 1 | 16 | 16 | 1 | 1 | 1110.08 | 0.7 |
| Rifampin | 16 | 0.5 | 16 | 0.5 | 32 | 32 | 1 | 1 | 822.94 | 2.76 |
| Vancomycin | 2048 | 128 | 1024 | 64 | 16 | 16 | 2 | 2 | 1449.25 | −4.86 |
| Ampicillin | 256 | 16 | 64 | 4 | 16 | 16 | 4 | 4 | 349.405 | −2.26 |
| Levofloxacin | 0.125 | 0.063 | 0.031 | 0.004 | 2 | 8 | 4 | 16 | 361.37 | −0.28 |
| Ciprofloxacin | 0.063 | 0.031 | 0.016 | 0.004 | 2 | 4 | 4 | 8 | 331.34 | −0.81 |
| Nalidixic acid | 64 | 32 | 8 | 2 | 2 | 4 | 8 | 16 | 232.24 | −0.25 |
| Chloramphenicol | 8 | 2 | 1 | 0.125 | 4 | 8 | 8 | 16 | 323.13 | 0.69 |
| Triclosan | >1024 | >1024 | 32 | 8 | UD | 4 | 32 | 128 | 289.54 | 4.8 |
| Erythromycin | 128 | 2 | 16 | 0.125 | 64 | 128 | 8 | 16 | 733.93 | 1.57 |
| Azithromycin | 128 | 4 | 4 | 0.063 | 32 | 64 | 32 | 64 | 748.98 | −1.23 |
| Novobiocin | 512 | 64 | 32 | 1 | 8 | 32 | 16 | 64 | 612.62 | 1.36 |
| Tetracycline | 4 | 0.5 | 2 | 0.031 | 8 | 64 | 2 | 16 | 467.52 | −13.63 |
| SDS | >10000 | 10000 | 10000 | 156.3 | ≧2 | 64 | ≧2 | 64 | 288.38 | 2.04 |
| Cloxacillin | >2048 | 512 | 128 | 8 | ≧4 | 16 | ≧8 | 64 | 435.88 | −0.98 |
| Carbenicillin | 32 | 2 | 0.5 | 0.063 | 16 | 8 | 64 | 32 | 378.4 | −5.91 |

Thus, using the controlled permeabilization of E. coli and P. aeruginosa outer membranes, one can delineate the specific contributions of active efflux and passive uptake to the activity of a given antibiotic.

Permeabilization of the Outer Membrane Increases Hit Rates and Diversity of Active Compounds in Drug Screening Assays.

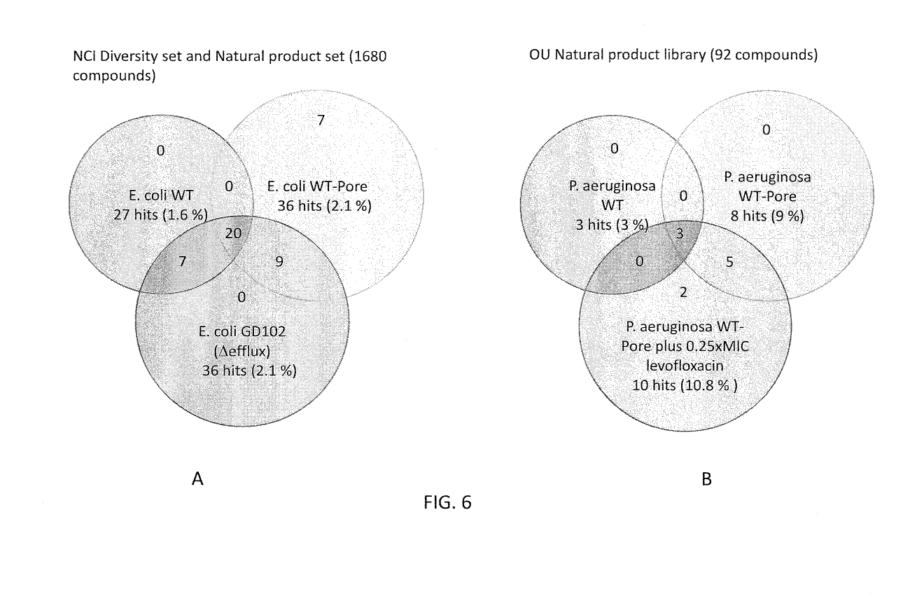

To evaluate possible applications of constructed strains in drug screening, two experiments were carried out. Results are represented in FIG. 6. In the first experiment, the NCI Diversity 5 set of 1563 compounds and the NCI Natural Product Set of 117 compounds were screened for potentiation of the antimicrobial activity of an antibiotic novobiocin in E. coli cells. In this screen, antibiotic novobiocin is present at sub-inhibitory (0.25×MIC) concentrations. Three strains were compared: the WT, the permeabilized WT-Pore and the efflux deficient ΔTolC cells. This screen identified 27 primary hits that potentiate the novobiocin antimicrobial activity in the WT cells (1.6% hit rate), whereas 36 hits (2.1% rate) were identified in screens using either the permeabilized WT-Pore or efflux-deficient ΔTolC cells. Importantly, in addition to increasing the hit rate to that in the efflux-deficient cells, the use of WT-Pore cells led to identification of 7 compounds that were uniquely active in these cells.

In the second experiment, P. aeruginosa PAO1 and PAO1-Pore cells were compared in screening of a small subset (92 fractions) of the OU Natural Product library to identify fractions with antimicrobial activities and fractions potentiating the antimicrobial activity of an antibiotic levofloxacin. Three fractions contained the anti-pseudomonal activity, which could be identified using the WT cells. In contrast, the anti-pseudomonal activity was detected in eight fractions when the permeabilized PAO1-Pore cells were used, an almost three times increase in the hit rate. Two additional fractions demonstrated the anti-pseudomonal activity against PAO1-Pore in a combination with a subinhibitory (0.25×MIC) concentration of levofloxacin. Thus, the permeabilized E. coli and P. aeruginosa are useful tools in screening of chemical and natural products libraries to identify compounds with novel antimicrobial and biological activities.

### DISCUSSION

The low permeability barrier of the outer membrane acting synergistically with active efflux significantly limits activities of antibiotics in Gram-negative cells [5-7]. The results presented here show that contributions from these two mechanisms of resistance could be defined through analyses of the set of strains differing in efflux capacities and permeabilities of the outer membranes.

When reconstituted into artificial lipid bilayers, the FhuAΔC/Δ4L protein pore, with the cross-sectional sides of 3.1×4.4 nm, was shown to have large conductance and possibly be ready for translocation of bulky biopolymers [8]. Here we demonstrated for the first time that such a large pore can be assembled in the outer membrane of E. coli and P. aeruginosa cells and that this pore efficiently removes the permeability barrier for compounds as large as ˜1450 Da (FIG. 3 and Tables 5 and 6). The expression of the pore is tightly controlled as seen from the inducer-dependent changes in the amounts of the protein and the susceptibilities of cells to antibiotics (FIG. 1 and Tables 5 and 6). The presence of the pore in the outer membrane does not significantly affect the physiology of growing cells (FIG. 2). The permeabilized cells grow with rates comparable to those of parental strains (FIG. 2) and their susceptibilities to aminoglycosides, activities of which are not affected by efflux and uptake, remain unchanged (Tables 5 and 6). Hence, the inducer-dependent changes in susceptibilities to antibiotics of the pore-producing cells mostly reflect the increased intracellular accumulation of antibiotics. Direct measurements of intracellular levels of erythromycin and azithromycin in E. coli and P. aeruginosa, respectively, strongly support this conclusion (FIGS. 4 and 5). It is possible however that for some antibiotics, physiological changes in cells due to the loss of efflux and an increased uptake could also contribute to a decrease of MICs observed in ΔTolC-Pore and Δ3-Pore cells (Tables 5 and 6).

Although MICs are not always a good measure of efflux capacities of cells [9], they often correlate with increased levels of intracellular accumulation of antibiotics. We found that among various tested antibacterial agents, only aminoglycosides remained insensitive to changes in active efflux and permeability of the outer membranes. This is surprising because the hydrophilic aminoglycosides are too large to diffuse through P. aeruginosa OprF with a cut-off size of ˜200 Da, and it was proposed that these antibiotics penetrate the outer membrane of P. aeruginosa by a self-promoted uptake through the interaction with and disruption of the LPS leaflet [10]. Our results however, strongly suggest that neither the outer membrane permeability, nor active efflux limit activities of these antibiotics.

Activities of all other tested compounds were potentiated by either the inactivation of efflux pumps, the presence of the pore, or both (Tables 5 and 6). Furthermore, in screening of the test libraries of compounds, we found significant increase in the hit rate and diversity of the active compounds (FIG. 6).

In at least certain embodiments, the present disclosure is directed to a mutant of a Gram-negative bacterium, the mutant comprising an outer membrane comprising at least one modified FhuA nanopore absent an N-terminal plug domain. The outer membrane may have an enhanced permeability to an antibiotic which is at least ten-fold greater than an outer membrane permeability to said antibiotic in a strain of the Gram-negative bacterium comprising a wild type FhuA nanopore. The modified FhuA nanopore may be absent four external loops. The modified FhuA nanopore may be FhuA ΔC/Δ4L protein. The mutant may comprise a mutation in at least one of the following genes: acrB, acrD, acrEF, emrB, emrY, entS, macB, mdtC, mdtF, tolC, mdfA, emrE, norM, mexAB-oprM, mexCD-oprJ, mexEF-oprN, mexXY, mexGHI, triABC, opmH, mexJKL, adeAB, adeFGH, adeIJK, amrRAB-oprA, bpeAB-oprB, bpeEF-oprC, ompF, ompC, oprD, ompA, carO, oprD, opcP1, and opcP2. The enhanced permeability of the mutant may be at least 100-fold greater than the outer membrane permeability in the strain of the Gram-negative bacterium comprising a wild type FhuA nanopore. The antibiotic may be selected from the group consisting of Amikacin, Gentamicin, Streptomycin, Levofloxacin, Nalidixic acid, Lincomycin, Chloramphenicol, Triclosan, Tetracycline, Ciprofloxacin, Proflavine, SDS, Cloxacillin, Carbenicillin, Ampicillin, Coumermycin, Rifampicin, Vancomycin, Erythromycin, Azithromycin, Virginiamycin, Novobiocin, and Tobramycin. The mutant may comprise an increased sensitivity to at least one compound of the group consisting of Amikacin, Gentamicin, Streptomycin, Levofloxacin, Nalidixic acid, Lincomycin, Chloramphenicol, Triclosan, Tetracycline, Ciprofloxacin, Proflavine, SDS, Cloxacillin, Carbenicillin, Ampicillin, Coumermycin, Rifampicin, Vancomycin, Erythromycin, Azithromycin, Virginiamycin, Novobiocin, and Tobramycin, as compared to a wild-type version of the Gram-negative bacterium. The increased sensitivity may be measured as a decrease in minimum inhibitory concentration. The decrease in minimum inhibitory concentration may be at least 10-fold, at least 100-fold, or at least 1000-fold.

In at least certain embodiments, the present disclosure is directed to a screening method for identifying a compound having an anti-bacterial activity, comprising (1) providing a mutant of a Gram-negative bacterium, the mutant comprising an outer membrane comprising at least one modified FhuA nanopore absent an N-terminal plug domain, (2) exposing the mutant to a test compound under conditions suitable for growth of the mutant; and (3) identifying the test compound as a possible drug candidate against said Gram-negative bacterium when the test compound inhibits growth of the mutant. The outer membrane of the mutant may have an enhanced permeability to an antibiotic which is at least ten-fold greater than an outer membrane permeability to said antibiotic in a strain of the Gram-negative bacterium comprising a wild type FhuA nanopore. The modified FhuA nanopore of the mutant may be absent four external loops. The modified FhuA nanopore of the mutant may be FhuA ΔC/Δ4L protein. The mutant may comprise a mutation in at least one of the following genes: acrB, acrD, acrEF, emrB, emrY, entS, macB, mdtC, mdtF, tolC, mdfA, emrE, norM, mexAB-oprM, mexCD-oprJ, mexEF-oprN, mexXY, mexGHI, triABC, opmH, mexJKL, adeAB, adeFGH, adeIJK, amrRAB-oprA, bpeAB-oprB, bpeEF-oprC, ompF, ompC, oprD, ompA, carO, oprD, opcP1, and opcP2. The enhanced permeability of the mutant may be at least 100-fold greater than the outer membrane permeability in the strain of the Gram-negative bacterium comprising a wild type FhuA nanopore.

It will be understood from the foregoing description that various modifications and changes may be made in the various embodiments of the present disclosure without departing from their true spirit. The description provided herein is intended for purposes of illustration only and is not intended to be construed in a limiting sense. Thus, while the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus the examples described above, which include particular embodiments, will serve to illustrate the practice of the present disclosure, it being understood that the particulars shown are by way of example and for purposes of illustrative discussion of particular embodiments of the present disclosure only and are presented in the cause of providing what is believed to be a useful and readily understood description of procedures as well as of the principles and conceptual aspects of the inventive concepts. Changes may be made in the formulation of the various components and compositions described herein, the methods described herein or in the steps or the sequence of steps of the methods described herein without departing from the spirit and scope of the present disclosure. All patents, published patent applications, and non-patent publications referenced in any portion of this application are herein expressly incorporated by reference in their entirety to the same extent as if each individual patent or publication was specifically and individually indicated to be incorporated by reference.

### REFERENCES

1. Tikhonova, E. B., Q. Wang, and H. I. Zgurskaya, Chimeric Analysis of the Multicomponent Multidrug Efflux Transporters from Gram-Negative Bacteria. J Bacteriol, 2002. 184(23): p. 6499-6507.
2. Choi, K.-H. and H. P. Schweizer, mini-Tn7 insertion in bacteria with secondary, non-glmS-linked attTn7 sites: example Proteus mirabilis HI4320. Nat. Protocols, 2006. 1(1): p. 170-178.
3. Cai, H., K. Rose, L. H. Liang, S. Dunham, and C. Stover, Development of a liquid chromatography/mass spectrometry-based drug accumulation assay in Pseudomonas aeruginosa. Anal Biochem, 2009. 385(2): p. 321-5.
4. Schurek, K. N., A. K. Marr, P. K. Taylor, I. Wiegand, L. Semenec, B. K. Khaira, and R. E. Hancock, Novel genetic determinants of low-level aminoglycoside resistance in Pseudomonas aeruginosa. Antimicrob Agents Chemother, 2008. 52(12): p. 4213-9.
5. Zgurskaya, H. I., C. A. López, and S. Gnanakaran, Permeability Barrier of Gram-Negative Cell Envelopes and Approaches To Bypass It. ACS Infectious Diseases, 2015. 1(11): p. 512-522.
6. Nikaido, H., Preventing drug access to targets: cell surface permeability barriers and active efflux in bacteria. Semin Cell Dev Biol, 2001. 12(3): p. 215-23.
7. Nikaido, H. and J. M. Pages, Broad-specificity efflux pumps and their role in multidrug resistance of Gram-negative bacteria. FEMS Microbiol Rev, 2012. 36(2): p. 340-63.
8. Mohammad, M. M., K. R. Howard, and L. Movileanu, Redesign of a Plugged β-Barrel Membrane Protein. Journal of Biological Chemistry, 2011. 286(10): p. 8000-8013.
9. Nagano, K. and H. Nikaido, Kinetic behavior of the major multidrug efflux pump AcrB of Escherichia coli. Proc Natl Acad Sci USA, 2009. 106: p. 5854-8.
10. Loh, B., C. Grant, and R. E. Hancock, Use of the fluorescent probe 1-N-phenylnaphthylamine to study the interactions of aminoglycoside antibiotics with the outer membrane of Pseudomonas aeruginosa. Antimicrobial Agents and Chemotherapy, 1984. 26(4): p. 546-551.

What is claimed is:

1. A mutant of a Gram-negative bacterium, the mutant comprising an outer membrane comprising at least one modified FhuA nanopore absent an N-terminal plug domain.

2. The mutant of claim 1, wherein the outer membrane has an enhanced permeability to an antibiotic which is at least ten-fold greater than an outer membrane permeability to said antibiotic in a strain of the Gram-negative bacterium comprising a wild type FhuA nanopore.

3. The mutant of claim 1, wherein the modified FhuA nanopore is absent four external loops.

4. The mutant of claim 1, wherein the modified FhuA nanopore is FhuA ΔC/Δ4L protein.

5. The mutant of claim 1, further comprising a mutation in at least one of the following genes: acrB, acrD, acrEF, emrB, emrY, entS, macB, mdtC, mdtF, tolC, mdfA, emrE, norM, mexAB-oprM, mexCD-oprJ, mexEF-oprN, mexXY, mexGHI, triABC, opmH, mexJKL, adeAB, adeFGH, adeIJK, amrRAB-oprA, bpeAB-oprB, bpeEF-oprC, ompF, ompC, oprD, ompA, carO, oprD, opcP1, and opcP2.

6. The mutant of claim 2, wherein the enhanced permeability of the mutant is at least 100-fold greater than the outer membrane permeability in the strain of the Gram-negative bacterium comprising a wild type FhuA nanopore.

7. The mutant of claim 2, wherein the antibiotic is selected from the group consisting of Amikacin, Gentamicin, Streptomycin, Levofloxacin, Nalidixic acid, Lincomycin, Chloramphenicol, Triclosan, Tetracycline, Ciprofloxacin, Proflavine, SDS, Cloxacillin, Carbenicillin, Ampicillin, Coumermycin, Rifampicin, Vancomycin, Erythromycin, Azithromycin, Virginiamycin, Novobiocin, and Tobramycin.

8. The mutant of claim 1, comprising an increased sensitivity to at least one compound of the group consisting of Amikacin, Gentamicin, Streptomycin, Levofloxacin, Nalidixic acid, Lincomycin, Chloramphenicol, Triclosan, Tetracycline, Ciprofloxacin, Proflavine, SDS, Cloxacillin, Carbenicillin, Ampicillin, Coumermycin, Rifampicin, Vancomycin, Erythromycin, Azithromycin, Virginiamycin, Novobiocin, and Tobramycin, as compared to a wild-type version of the Gram-negative bacterium.

9. The mutant of claim 8, wherein the increased sensitivity is measured as a decrease in minimum inhibitory concentration.

10. The mutant of claim 8, wherein the decrease in minimum inhibitory concentration is at least 10-fold.

11. The mutant of claim 8, wherein the decrease in minimum inhibitory concentration is at least 100-fold.

12. The mutant of claim 8, wherein the decrease in minimum inhibitory concentration is at least 1000-fold.

13. A screening method for identifying a compound having an anti-bacterial activity, comprising:
providing a mutant of a Gram-negative bacterium, the mutant comprising an outer membrane comprising at least one modified FhuA nanopore absent an N-terminal plug domain;
exposing the mutant to a test compound under conditions suitable for growth of the mutant; and
identifying the test compound as a possible drug candidate against said Gram-negative bacterium when the test compound inhibits growth of the mutant.

14. The screening method of claim 13, wherein the outer membrane of the mutant has an enhanced permeability to an antibiotic which is at least ten-fold greater than an outer membrane permeability to said antibiotic in a strain of the Gram-negative bacterium comprising a wild type FhuA nanopore.

15. The screening method of claim 13, wherein the modified FhuA nanopore of the mutant is absent four external loops.

16. The screening method of claim 13, wherein the modified FhuA nanopore of the mutant is FhuA ΔC/Δ4L protein.

17. The screening method of claim 13, wherein the mutant further comprises a mutation in at least one of the following genes: acrB, acrD, acrEF, emrB, emrY, entS, macB, mdtC, mdtF, tolC, mdfA, emrE, norM, mexAB-oprM, mexCD-oprJ, mexEF-oprN, mexXY, mexGHI, triABC, opmH, mexJKL, adeAB, adeFGH, adeIJK, amrRAB-oprA, bpeAB-oprB, bpeEF-oprC, ompF, ompC, oprD, ompA, carO, oprD, opcP1, and opcP2.

18. The screening method of claim 14, wherein the enhanced permeability of the mutant is at least 100-fold greater than the outer membrane permeability in the strain of the Gram-negative bacterium comprising a wild type FhuA nanopore.
